(19) United States
(12) Patent Application Publication
(10) Pub. No.: US 2017/0056319 A1
(43) Pub. Date: Mar. 2, 2017

(54) Positive pressure capsules and method of manufacturing the same

(71) Applicants: GARRY TSAUR, Rowland Heights, CA (US); TING-HUA WANG, Taichung City (TW)

(72) Inventors: GARRY TSAUR, Rowland Heights, CA (US); TING-HUA WANG, Taichung City (TW)

(21) Appl. No.: 14/839,206

(22) Filed: Aug. 28, 2015

(51) Int. Cl. A61K 9/00; A61K 9/48

(52) U.S. Cl. CPC A61K 9/0004; A61K 9/4808; A61K 9/4833

(57) ABSTRACT

In a positive pressure capsule and its manufacturing method, it includes injection of a drug through at the opening of the capsule followed by sealing the capsule and internal of the capsule contains air space so as to form a positive pressure capsule. The features of the positive pressure capsule are adhesion seams and liquid drugs can be filled inside such capsule. The capsule may be a hard capsule and the raw material of the capsule may be gelatin or HPMC.

### BACKGROUND OF THE INVENTION

1. Field of the Invention

The present invention relates to a positive pressure capsule and method of manufacturing of said capsule, the capsule can accelerate the destruction of the capsule and form an opening and help quick release of drug. Said capsule is characterized with a sealed capsule containing a gas and the internal pressure of the capsule is maintained at a positive pressure.

2. Description of the Prior Art

Capsule is a fast developing pharmaceutical dosage form and 0.1˜0.7 mm capsules are widely used in oral drugs and health foods with the advantages of easy storage, accurate dose and controllable drug release; moreover, capsule can mask the odor of the active ingredients of drugs and maintain drug activity and therefore are easier for the users to accept.

The dosage forms of capsules allow storage of drugs inside capsules and the sealing characteristic of capsules helps prevent reduced drug activity and keep moisture away from the contained drug by avoiding direct contact of the contained drug contact with outside air and water and as a result helps maintain long-term stability.

Some pharmacologically active substances may be difficult to be formulated into commercially acceptable formulations due to their biopharmaceutical or physico-chemical properties. However, such substances may be administered in the form of a liquid carrier medium and the solvents 1,2-propanediol as well as dimethylisosorbide both have great potential to be used as a liquid carrier medium. The composition of carrier media allows formation of an emulsion in the stomach, thereby facilitating the absorption of the pharmaceutically active substance. A carrier medium must be accurately prepared, so that it does not affect the pharmaceutical composition and does not destroy its beneficial properties. Solubilizing properties of the drug carrier may change the nature of the active substance and lead to precipitation, resulting in insufficient dose in patients. The emulsifying properties of pharmaceutical carriers may vary and may not form an emulsion in the stomach upon administration and a pharmaceutically active substance will not be absorbed properly.

Such liquid formulations encapsulated in the capsule provide a very convenient method for administration of such pharmaceutically active substances. However, commercially acceptable liquid-filling capsules are also accompanied by difficulties in limitations on applications.

Capsules used in pharmaceutical products can be divided into hard capsules and soft capsules, including hard capsules (hereinafter referred to as hard capsules) which can be filled with powder or fine particles of solid pharmaceutical drugs and soft capsules (hereinafter referred to as soft capsules) which can be filled other liquid drugs.

The raw materials of capsules can be gelatin, sorbitol, hydroxypropyl methylcellulose (HPMC) and opaque food coloring or other additives.

Traditional raw material for producing capsules is gelatin which is a protein or peptide extracted from animal skin, bone, or connective tissues and contains collagen and a hydrophilic layer of pale yellow protein layer is obtained after boiling in the water, namely gelatin.

Another commonly raw material of capsules is hydroxypropyl methylcellulose (HPMC), which is a polymer with viscosity and often used as a replacement for excipients, emulsifiers, thickeners, suspending agents or gelatin substitute in oral drugs. After alkali treatment, the alkoxy anion produced by hydroxy deprotonation of cellulose can act on propylene oxide and generate hydroxypropyl cellulose ether as well as be condensed with methyl chloride to form methyl cellulose ether. In addition, hydroxypropyl methyl cellulose will be produced if both reactions occur simultaneously.

Other alternative replacements of the raw materials of capsules are also available, including seaweed extracts i.e. polysaccharide hydrocolloid as an alternative raw material. Plant materials help prevent cross-linking reaction with proteins of animal source and maintain the strength of the capsule body. Furthermore, because of recent BSE epidemic in the U.S., improvement of the raw materials of soft capsules has been taken seriously in recent years. The market share of other alternative raw materials, such as agarose, soluble starch ester derivatives, HPMC or carragheenin, also has increased gradually over the years.

One of the purposes of improvement of capsules is to enhance the bioavailability of pharmaceutical agents. The active ingredient can be released quickly in the form of a liquid when the capsule ruptures. And different from lozenge composition, complete disintegration of the capsule is not necessary to make the active ingredient become absorbable. Meanwhile, dispersion of the insoluble active ingredients in liquid carriers provides faster absorption, e.g. a solution of a hydrophobic drug in a hydrophilic solvent; while the capsule will be broken in the stomach during the process of digestion and hydrophobic drugs may be dispersed in gastric juice.

Nonetheless, slow disintegration of capsules is a common problem and studies have suggested that the slow disintegration of capsules was mainly due to crosslinking of gelatin. Since the amino acids in gelatin molecules are prone to produce aldehyde by oxidation, crosslinking reaction may occur between the generated aldehyde and surrounding amino acids, thereby forming a intramolecular/intermolecular three-dimensional network structure and result in increase of the molecular weight and structure stability as well as reduced solubility of gelatin which consequently cause slow disintegration of capsules. For the problem of capsule disintegration, the solutions include: 1. addition of antioxidants, 2 adjustment capsule formula and change its composition, such as the ratio of water and elasticizer, 3 uses materials that are less easily oxidized.

### SUMMARY OF THE INVENTION

To solve the aforementioned problem of difficult disintegration of capsules that affecting the bioavailability of the drug in gastric fluid, the inventors have developed the present invention.

In one aspect, present invention provides a positive capsule which takes advantage of the internal positive pressure of the capsule and accelerates destruction and disintegration of the capsule in the stomach after administration and subsequently accelerates release of the drug so as to facilitate drug absorption by organisms.

According to the invention, the capsule is a sealed seamless capsule.

According to the invention, the internal pressure of the capsule must be a positive pressure and is higher than one atmosphere pressure.

According to the invention, the capsule contains a gas and the gas is oxygen, helium, nitrogen, or carbon dioxide or a mixture of the abovementioned gases.

According to the invention, the raw material of the capsule is gelatin, that is, animal source hydrolyzed collagen.

According to the invention, the raw material of the capsule is hydroxypropyl methylcellulose (HPMC).

According to the invention, the capsule material may further include agar, starch, alginic acid, guar gum, and a pharmaceutically acceptable sunscreen, plasticizer and the like.

According to the invention, the gelatin is alkali-treated gelatin, acid-treated gelatin, or chemically modified gelatin.

According to the invention, the plasticizer is selected from the following groups: glycerin, sorbitol, maltose, glucose, polysaccharides, sucrose, xylitol, mannitol, propylene glycol, polyethylene glycol.

According to the invention, the sunscreen agent is selected from the following groups: caramel, titanium oxide, iron oxide.

According to the invention, the filling of the capsule is liquid, suspension, paste, powder, granule and the like.

According to the invention, the present invention provides a method for producing positive pressure capsules, wherein the capsules are filled under positive pressure environment followed by sealing the capsule while the bubbles in the capsule are retained and such capsule product is a positive pressure capsule under the environment of 1 atm.

According to the invention, the present invention also provides another method for producing positive pressure capsules, wherein the capsules are filled at low temperature and the temperature is below 25° C. followed by sealing the capsule while a bubble is retained inside the capsule and such capsule product is a positive pressure capsule at room temperature.

### BRIEF DESCRIPTION OF THE DRAWINGS

FIG. 1 is the schematic diagram of the positive pressure capsule of the invention.

FIG. 2 is another schematic diagram of the positive pressure capsule of the invention.

FIG. 3A is the schematic diagram of the positive pressure capsule of the invention in the stomach before been digested after administration.

FIG. 3B is the schematic diagram of the positive pressure capsule of the invention in the stomach under digestion after administration.

### DETAILED DESCRIPTION OF THE PREFERRED EMBODIMENT

All technical and scientific terms used in the invention, unless otherwise specified, have the common meanings that are understood by person skilled in the art. The foregoing detailed description of the invention and the specific examples are provided herein for the purpose of illustration only, and the invention is not limited to the preferred embodiments shown. It should be understood that any changes or modifications within the spirit of the invention shall be included in the scope of present invention.

The purpose of present invention is to provide a method of manufacturing a positive pressure capsule and said method comprises following steps:

Step 1: place the capsule to be filled in a positive pressure or low temperature environment;

Step 2: fill the capsule with drug and retain the internal space containing bubbles;

Step 3: seal the capsule and ensure no opening is found on the surface to prevent gas leak.

The positive pressure environment mentioned above refers to the environment with a more than one atmosphere pressure and one atmosphere pressure is equivalent to the international unit 1 atm, 1013 hPa, or 76 cm-Hg.

The bubbles mentioned above shall be retained to a level that does not affect drug administration dose and limitation on the size of the bubble is not necessary; in addition, said gas shall not affect the drug and content enclosed.

The low temperature environment mentioned above refers to a temperature lower than 25° C.

The shape of the positive pressure capsule mentioned above can be spherical, oval, long oval, tubular and polyhedral capsule and though the size of said capsule is not specified the content shall be adjusted to within the range from 1 mg to 10 g.

The raw material of the capsule of the invention is a reversible gelling substance, even after drying. Said substance, for example, made by HPMC, gelatin, agar, starch, alginic acid and guar gum, and is preferably a material containing gelatin or HPMC and a plasticizer. Moreover, the materials of the capsule may include additives such as a sunscreen, if necessary.

The gelatin mentioned above refers to gelatin obtained from hydrolyzed collagen extracted from animals such as cattle and pig. Also, to be used as the reversible gelling substance, the gelatin must be subjected to further treatments including alkali treatment, acid treatment or chemical modification. Acid treatment means hydrochloric acid or sulfuric acid is used as the hydrolytic agent; alkali treatment means alkaline salts such as lime is used as the hydrolytic agent; and chemically modified gelatin refers to gelatin obtained after the amino group of the gelatin reacts with organic acids such as succinic acid or phthalic acid.

The plasticizer mentioned above includes but is not limited to glycerol, sorbitol, maltose, glucose, polysaccharides, sucrose, xylitol, mannitol, propylene glycol, and polyethylene glycol and the like.

The sunscreen mentioned above includes but is not limited to caramel, titanium oxide, iron oxide and the like.

The form of the capsule filling of the invention is not specified and can be in liquid, suspension, paste, powder or granule.

The thickness of the hard capsule of the invention is not subject to any limitation only if it is thick enough to allow the capsule to exert its function. However, a thickness between 0.01 to 5 mm is better and the preferable thickness is 0.05-1 mm.

The capsule of the invention can be used for production of pharmaceutical products, pharmaceutical product under registration, food and cosmetics, based on the composition of the filling.

No limitation is specified for the pharmaceutical products and pharmaceutical product under registration mentioned above if the active ingredient of the capsule of the invention does not damage the function of the capsule. The pharmaceutical products include but are not limited to the following products: vitamins, antipyretics, analgesics, anti-inflammatory agents, anti-ulcer agents, cardiotonics, anticoagulants, hemostatic agents, anti-resorptive agents, inhibiting angiogenesis agents, antidepressants, anti-tumor agents, antitussives, muscle relaxants, antiepileptic agents, anti-allergic agent, arrhythmia therapeutic agents, vasodilators, antihypertensive diuretics, therapeutic agents for diabetes, anti-tuberculosis agents, hormones, anti-bacterial agents, anti-fungicides and anti-viral agents but are not limited to the abovementioned groups of pharmacological action and all active ingredients that have relatively poor solubility in water are the objects of this invention. Active insoluble substances are preferred.

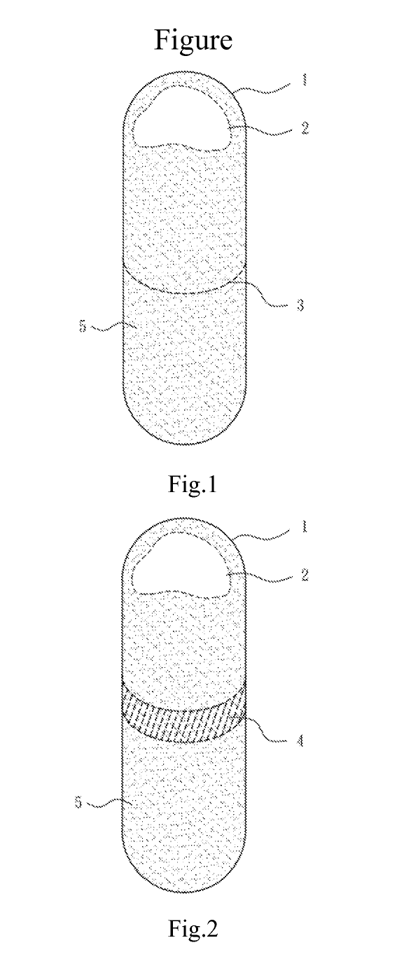

The present invention will now be described more specifically with reference to the schematic diagram of FIG. 1 and FIG. 2:

### Example 1

The method for manufacturing the positive capsule provided in the invention comprises following steps:

Step 1: place the capsule in a positive pressure environment and the environment shall have more than 1 atmosphere of pressure;

Step 2: under the positive pressure condition, fill the capsule with drug (5);

Step 3: seal the capsule body (1) and ensure no opening is found on the surface to prevent gas leak, but retain the bubbles (2) inside.

Step 4: return the capsule to normal environment, i.e. 1 atmosphere of pressure and becomes a positive pressure capsule.

The positive pressure capsule mentioned above, before or when sealing the capsule body (1), a gas can be injected to allow the air room contain the injected gas (2) followed by sealing the capsule.

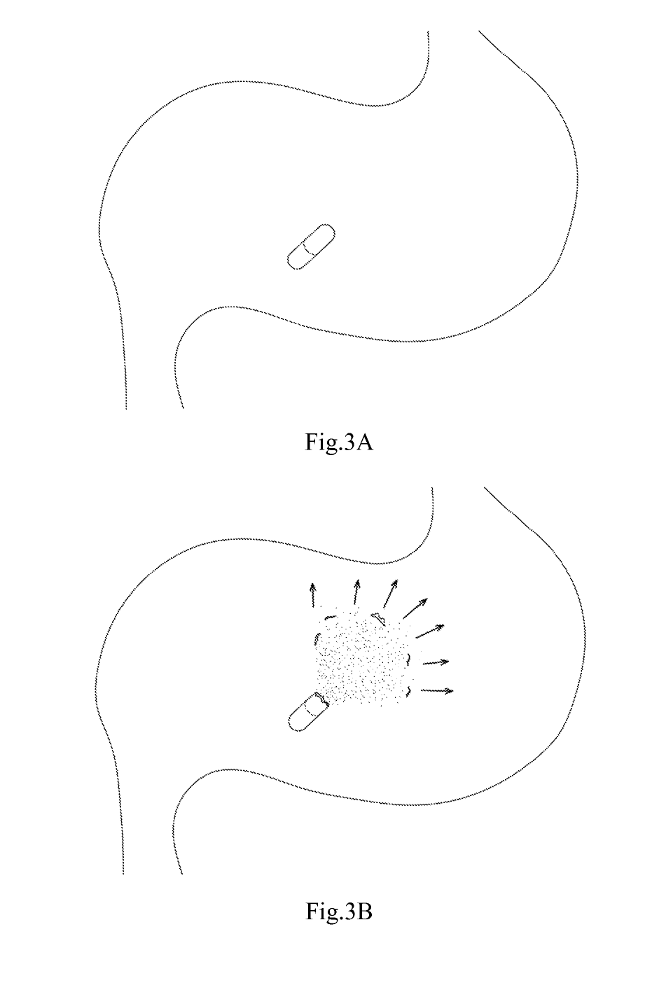

The positive pressure capsule, after ingestion by the user and when entering stomach for digestion, will release the drug (5) contained when the capsule is destroyed to a certain degree and the positive pressure is sufficient to break the capsule and form an opening (FIG. 3A and FIG. 3B).

### Example 2

Step 1: place the capsule in a low temperature environment and the low temperature condition shall be lower than 25° C.;

Step 2: under the low temperature condition, fill the capsule with drug (5);

Step 3: seal the capsule body (1) and ensure no opening is found on the surface to prevent gas leak, but retain the bubbles (2) inside.

Step 4: return the capsule to normal environment, i.e. room temperature, and the internal of the capsule will have more than 1 atmosphere of pressure and becomes a positive pressure capsule.

The positive pressure capsule mentioned above, before or when sealing the capsule body (1), a gas can be injected to allow the air room contain the injected gas (2) followed by sealing the capsule.

The positive pressure capsule, after ingestion by the user and when entering stomach for digestion, will release the drug (5) contained when the capsule is destroyed to a certain degree due to increased pressure caused by body temperature and the positive pressure is sufficient to break the capsule and form an opening (FIG. 3A and FIG. 3B).

The capsule is a sealed capsule and the capsule must be a sealed capsule body (1). The method for sealing capsules can be the adhesion line (3) on the capsule body shown in FIG. 1, or the adhesion area (4) on the capsule body shown in FIG. 2 and said adhesion area is a sealed area and is formed after partial melting of the capsule body. Nonetheless, present invention is not limited to the method for capsule sealing for manufacturing sealed capsules and the embodiments are provided for the purpose of demonstration that the positive pressure capsule of the invention must be a sealed capsule rather than limitation.

The foregoing detailed description of the invention and the specific examples are provided herein for the purpose of illustration only, and the invention is not limited to the preferred embodiments shown. It should be understood that any changes or modifications within the spirit of the invention shall be included in the scope of present invention.

In summary, present invention not only provides a novel method, but also discloses a number of improved features of technologies as described above. Therefore, the invention meet the requirements of novelty as well as non-obviousness (Article 33(2) PCT or 33PCT(2)). The applicant respectfully requests a favor on approval of the patent.

1. A positive pressure capsule, comprising:
a hollow body;
fillings in the hollow body; and
bubbles, being set within the body;
wherein a pressure of the bubble is higher than 1 atm.

2. The positive pressure capsule as recited in claim 1, wherein the shape of the body is spherical, oval, long oval, tubular or polyhedral capsule.

3. The positive pressure capsule as recited in claim 1, wherein gas contained in the bubble is oxygen, helium, nitrogen, or carbon dioxide.

4. The positive pressure capsule as recited in claim 1, wherein the raw material of the body is gelatin or hydroxypropyl methylcellulose (HPMC).

5. The positive pressure capsule as recited in claim 1, wherein the raw material of the body further includes agar, starch, alginic acid, guar gum, pharmaceutical acceptable additives, wherein the pharmaceutical acceptable additives include plasticizers or sunscreens.

6. The positive pressure capsule as recited in claim 4, wherein the gelatin is alkali-treated gelatin, acid-treated gelatin, or chemically modified gelatin.

7. The positive pressure capsule as recited in claim 5, wherein the plasticizer is selected from the groups consisting of glycerol, sorbitol, maltose, glucose, polysaccharides, sucrose, xylitol, mannitol, propylene glycol, and polyethylene glycol.

8. The positive pressure capsule as recited in claim 5, wherein the sunscreen is selected from the groups consisting of caramel, titanium oxide, and iron oxide.

9. The positive pressure capsule as recited in claim 1, wherein the filling of the capsule is a pharmaceutical composition or health food in the form of liquid, suspension, paste, powder, or granule.

10. A method for manufacturing a positive capsule comprising of the following steps:
Step 1: placing the capsule in a positive pressure environment, wherein the environment shall have more than 1 atmosphere of pressure;
Step 2: under the positive pressure condition, filling the capsule with drug;
Step 3: sealing the capsule body and ensuring no opening is found on the surface to prevent gas leak, but retain the bubbles inside;
Step 4: returning the capsule to normal environment at 1 atmosphere of pressure, and becoming a positive pressure capsule.

11. A method for manufacturing a positive capsule comprising of the following steps:
Step 1: placing the capsule in a low temperature environment, wherein the low temperature condition must be lower than 25° C.;
Step 2: under the low temperature condition, filling the capsule with drug;
Step 3: sealing the capsule body and ensuring no opening is found on the surface to prevent gas leak, but retain the bubbles inside.
Step 4: returning the capsule to normal environment at room temperature, wherein the internal pressure of the capsule will have more than 1 atmosphere of pressure and becomes a positive pressure capsule.
